(19) United States
(12) Patent Application Publication
(10) Pub. No.: US 2026/0345834 A1
(43) Pub. Date: Nov. 5, 2026

(54) NIR-LIGHT ACTIVATABLE NANOGEL DELIVERY SYSTEM

(71) Applicant: THE BOARD OF TRUSTEES OF THE UNIVERSITY OF ILLINOIS, Urbana, IL (US)

(72) Inventors: Jefferson Kar Fai CHAN, Savoy, IL (US); Rodrigo TAPIA HERNANDEZ, Champaign, IL (US)

(73) Assignee: THE BOARD OF TRUSTEES OF THE UNIVERSITY OF ILLINOIS, Urbana, IL (US)

(21) Appl. No.: 19/103,623

(22) PCT Filed: Aug. 21, 2023
(86) PCT No.: PCT/US2023/072569

§ 371 Date: Feb. 13, 2025

Related U.S. Application Data
(60) Provisional application No. 63/399,702, filed on Aug. 21, 2022.

(51) Int. Cl. A61K 49/00; A61K 9/06; A61K 31/167; A61K 31/337; A61K 31/5377; A61K 33/243; A61P 35/00

(52) U.S. Cl. CPC A61K 49/0032; A61K 9/06; A61K 31/167; A61K 31/337; A61K 31/5377; A61K 33/243; A61P 35/00

(57) ABSTRACT

Herein is described the development of NIR-nanogels for NIR light-mediated cargo delivery. NIR-nanogels feature a photolabile cyanine crosslinker that can cleave via dioxetane chemistry when irradiated. This photochemical process results in the formation of two carbonyl fragments and concomitant NIR-nanogel degradation to facilitate cargo release. In contrast to studies where cyanines are utilized as photocages, our approach does not require direct chemical attachment to the cargo, thus expanding our ability to deliver molecules that cannot be covalently modified. We showcase this feature by encapsulating a palette of small-molecule chemotherapeutics that feature structurally diverse chemical architecture.

### RELATED APPLICATIONS

This application claims priority under 35 U.S.C. § 119(e) to U.S. Provisional Patent Application No. 63/399,702, filed Aug. 21, 2022, which is incorporated herein by reference.

### GOVERNMENT SUPPORT

This invention was made with government support under 1R35GM133581-01 awarded by the National Institutes of Health. The United States Government has certain rights in the invention.

### BACKGROUND OF THE INVENTION

Since the discovery of the first cyanine in the mid-1800s, molecules belonging to this family of dyes have been employed as contrast agents in a diverse range of biomedical applications such as identification of blood vessels, sentinel lymph nodes, and tumors for fluorescence-guided surgery. Additionally, cyanines have served as important scaffolds for the development of activity-based sensing probes for in vivo molecular imaging. Some notable examples include sensors for metal ions, nitric oxide, pH, and reactive oxygen species (ROS). Beyond examples that exhibit absorbance and emission profiles in the visible and NIR range, the cyanine core can be readily diversified to access congeners that extend into the SWIR (NIR-II) window (>950 nm) to enable deeper tissue penetration. Despite their versatility and widespread use, cyanines in general, are limited by their flat planar structures which contribute to their poor aqueous solubility; reactivity toward nucleophiles (e.g., biological thiols) that can disrupt the conjugated x-system; propensity to undergo oxidative decomposition; and instability to extended light exposure.

Exploiting the ‘drawbacks’, such as those mentioned above, to design new chemical tools and approaches based on aberrant cyanine chemistry is an innovative way to turn a negative trait into a favorable property. For instance, cyanines that are prone to aggregate due to poor solubility can be induced to form self-assembled particles that can be used as red-shifted imaging agents. Likewise, the photoswitching behavior of cyanines that result from a thiol reversibly adding across the conjugated system is commonly employed for super-resolution microscopy. Moreover, fluorogenic probes for ratiometric detection of ROS such as O₂^{⋅-}, H₂O₂, ONOO⁻, ClO⁻, etc. have been developed by linking an unstable cyanine prone to oxidative cleavage to a reference dye that is resistant to ROS-mediated decomposition. Lastly, Schnermann and co-workers (J Am Chem Soc 2014, 136 (40), 14153-14159) have leveraged the photobleaching properties of heptamethine cyanine dyes to serve as NIR photocages, which can release an appended molecule (e.g., Gabapentin) through an intramolecular cyclization event after photolysis. However, it is notable that this uncaging strategy requires covalent appendage of the cargo to the cyanine photocage through a cleavable linker. As such, this approach is not suitable for molecules lacking a convenient handle for attachment.

Accordingly, there is a need for improved drug delivery systems that can specifically target a disease such as a cancer and deliver a chemotherapeutic to treat the disease.

### SUMMARY

To overcome the stated challenges above, we report the first cyanine-based nanogel system for NIR-light mediated drug delivery, which we have named NIR-nanogels. In addition to showcasing the generalizability of our approach by releasing structurally diverse cargo in vitro, we selected Paclitaxel for encapsulation to assess efficacy in a murine model of breast cancer.

Accordingly, this disclosure provides an NIR-light activatable nanogel comprising a crosslinked nanoparticle encapsulating a chemotherapeutic that is not covalently bound to the crosslinked nanoparticle, wherein the crosslinked nanoparticle comprises a cyanine of Formula I:

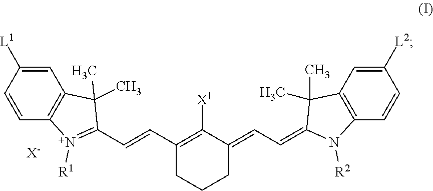

wherein
   L¹ and L² each independently comprise a crosslinking moiety;
   R¹ and R² are each independently —(C₁-C₆)alkyl;
   X¹ is halo; and
   X⁻ is a counter anion; and
a monomer crosslinked to L¹ and/or L².

This disclosure also provides a method for preparing an activatable nanogel described above, which method comprises:
a) forming an aqueous mixture comprising a surfactant, a compound of Formula I, and a monomer capable of crosslinking to L¹ and/or L² of Formula I;
b) degassing the aqueous mixture;
c) contacting the degassed aqueous mixture, a radical initiator, and a base to form a nanogel solution comprising a crosslinked nanoparticle;
d) purifying the nanogel solution; and
e) contacting the purified nanogel solution and a chemotherapeutic; wherein the activatable nanogel is thereby formed.

Additionally, this disclosure provides a method for treating cancer comprising:
a) administering to a subject having a cancer, an activatable nanogel described above, wherein the cancer uptakes a crosslinked nanoparticle of the activatable nanogel;
b) irradiating the cancer with near infrared light for a period of time sufficient to release a chemotherapeutic from the crosslinked nanoparticle; wherein the cancer is thereby treated.

The invention provides activatable nanogels comprising novel compounds of Formula I, intermediates for the synthesis of compounds of Formula I, as well as methods of preparing compounds of Formula I. The invention also provides activatable nanogels comprising compounds of Formula I that are useful as intermediates for the synthesis of other useful compounds. The invention provides for the use of compounds of Formula I for the manufacture of medicaments comprising activatable nanogels that are useful for the treatment of cancer in a mammal, such as a human.

The invention provides for the use of the compositions described herein for use in medical therapy. The medical therapy can be treating cancer, for example, breast cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Irradiation of intact nanogels with NIR light at 780 nm results in release of encapsulated cargo (smaller black dots). Cy780-Acryl crosslink shown as larger grey dots.

FIG. 2. Normalized absorbance spectra demonstrating time-dependent photobleaching of Cy780-Acetamido. Time points=0, 15, 30 mins. Samples were irradiated with a LED light source at 780 m, (300 mW power output, 47.3 μW/mm² maximum irradiance).

FIG. 3. Absorption measurements of a 25 μM solution of a) Cy7-Cl and b) Cy780-Acryl when irradiated with a 780 nm LED light. Time points=0, 15, 30 mins. Samples were irradiated with a LED light source at 780 m, (300 mW power output, 47.3 μW/mm² maximum irradiance).

FIG. 4. Absorption measurements of a NIR-nanogel sample without dissolved oxygen when irradiated with a 780 nm LED light. Time points=0, 15, 30, 45, 60, 75, 90 mins. Samples were irradiated with a LED light source at 780 m, (300 mW power output, 47.3 μW/mm² maximum irradiance).

FIG. 5A-B. HR-LCMS analysis of Cy780-Acryl (10 μM) in 2:48 v/v MeOH:H₂O upon irradiation with a 780 nm LED for 30 min. Products were separated on a CORTECS® UPLC C18 column (1.6 μm, 2.1 by 50 nm) with a linear gradient using a combination of solvent A (95% water 5% acetonitrile, 0.1% FA) and solvent B (95% acetonitrile, 5% water, 0.1% FA) at a flow rate of 0.4 mL/minute. Gradient protocol in minutes: 0-0.5 (95% A); 0.5-5 (30% A); 5-5.1 (100% B); 5.1-8 (100% B); 8-8.1 (95% A); and 8.1-10 (95% A). High-resolution spectra for the reaction products at elution times of a) 2.30 min; b) 2.69 min; c) 2.72 min; and d) 2.82 min.

FIG. 6. Dynamic light scattering intensity plot of NIR-nanogels, with an average diameter of 125±41 nm and a polydispersity index of 0.15.

FIG. 7. Representative SEM image of NIR-nanogels dried on a glass slide and sputter-coated with Au—Pd metal. Scale bar represents 500 nm.

FIG. 8A-F. a) Normalized absorbance spectra demonstrating time-dependent photobleaching of Cy780-Acryl. Time points=0, 15, 30, 45, 60, 90, 120, 150, 180 mins. b) Diameter of NIR-nanogels with and without NIR light irradiation measured via dynamic light scattering (left) and scanning electron microscopy (right). SEM images of NIR-nanogels c) before and d) after NIR light treatment for 30 mins. (n=300 for SEM) Scale bar represents 500 nm. e) Normalized absorbance of nanogels exposed to biologically relevant reactive species. f) Normalized absorbance of nanogels suspended in Britton-Robinson buffer (pH 4.2 to 12.0). Statistical analysis was performed using a two-tailed Student's t-test (α=0.05), ****P<0.0001.

FIG. 9. Normalized absorbance of NIR-nanogels when resuspended in PBS at t=0 and 60 minutes and when incubated with a 2% w/v solution of albumin bovine serum in PBS at t=0 and 60 minutes.

FIG. 10A-D. Passive release of encapsulated a) cisplatin b) gefitinib c) GW9662 and d) Paclitaxel via dialysis for 0, 1, 2, and 4 hours against DI water.

FIG. 11A-D. Photographs of 4T1 tumors excised from BALB/c mice after systemic treatment with NIR-nanogel (pac) a) without or b) with NIR light irradiation. Scale bars represents 1 cm. c) Tumor volume and d) tumor mass measurements of the tumors shown in a) and b). Statistical analysis was performed using a two-tailed Student's t-test (α=0.05), *P<0.05.

FIG. 12. Representative H&E staining of the heart, kidney, liver, spleen, and muscle tissue harvested from mice with and without NIR-nanogel (pac) treatment. Scalebar represents 50 μm.

FIG. 13. H&E staining of tumor tissues harvested from mice that were treated with NIR-nanogels loaded with paclitaxel (left) without NIR-light irradiation and (right) with NIR-light irradiation for a total of 30 minutes. Scalebar represents 50 μm.

FIG. 14. a) tumor volumes and b) tumor masses of mice treated with NIR-nanogels and irradiated with 780 nm LED light and mice treated with NIR-nanogels loaded with paclitaxel with and without 780 nm LED light irradiation. Statistical analysis was performed using a two-tailed Student's t-test (α=0.05), *P<0.05.

FIG. 15. Encapsulation of tacrine in nanogels analyzed by fluorescence spectroscopy.

FIG. 16. Preliminary imaging results of nanogels diffusing into brain (mice). Left: PA signal from NIR-nanogel spectrum (Light gray) after spectral unmixing. Right: Atlas reference to image frame.

FIG. 17. Preliminary imaging results of nanogels diffusing into brain (mice). Frame taken at a similar location/position of the brain of each mouse. Top: Photoacoustic imaging of a mouse's brain treated with NIR-nanogels via retro-orbital injection. Bottom: Photoacoustic imaging of a mouse's brain treated with saline via retro-orbital injection. Signal from nanogels appears as lighter gray/white.

### DETAILED DESCRIPTION

The favorable properties of cyanines (e.g., near-infrared (NIR) absorbance and emission) have made this class of dyes popular for a wide variety of biomedical applications. However, many cyanines are prone to rapid photobleaching when irradiated with light. In this study, we have exploited this undesirable trait to develop NIR-nanogels for NIR light-mediated cargo delivery. NIR-nanogels feature a photolabile cyanine crosslinker (Cy780-Acryl) that can cleave via dioxetane chemistry when irradiated. This photochemical process results in the formation of two carbonyl fragments and concomitant NIR-nanogel degradation to facilitate cargo release. In contrast to studies where cyanines are utilized as photocages, our approach does not require direct chemical attachment to the cargo, thus expanding our ability to deliver molecules that cannot be covalently modified. We showcase this feature by encapsulating a palette of small-molecule chemotherapeutics that feature structurally diverse chemical architecture. To demonstrate site-selective release in vivo, we generated a murine model of breast cancer. Relative to non-light irradiated and drug-free controls, treatment with NIR-nanogels loaded with paclitaxel (a potent cytotoxic agent) and NIR light resulted in significant attenuation of tumor growth. Moreover, we show via histological staining of the vital organs that minimal off-target effects are observed.

Additional information and data supporting the invention can be found in the following publication by the inventors: J. Am. Chem. Soc. 2022, 144, 18101-18108 and its Supporting Information, which are incorporated herein by reference in its entirety.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated.

Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl can be unsubstituted or optionally substituted with a substituent described below. For example, a phenyl moiety or group may be substituted with one or more substituents R^{X} where R^{X} is at the ortho-, meta-, or para-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light.

### Stated Embodiments of the Technology

1. A near infrared-light (NIR-light) activatable nanogel comprising a crosslinked nanoparticle encapsulating a chemotherapeutic that is not covalently bound to the crosslinked nanoparticle, wherein the crosslinked nanoparticle comprises a cyanine of Formula I:

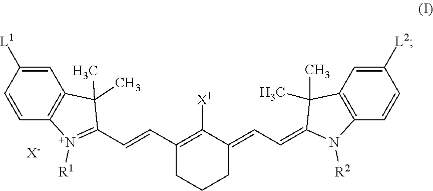

wherein
   L¹ and L² each independently comprise a crosslinking moiety;
   R¹ and R² are each independently —(C₁-C₆)alkyl;
   X¹ is halo; and
   X⁻ is a counter anion; and
a monomer crosslinked to L¹ and/or L², wherein the monomer comprises the crosslinking moiety.
2. The activatable nanogel of embodiment 1 wherein L¹ and L² are each independently —NR³(C═O)CR⁴═CHR⁵, —O(C═O)CR⁴═CHR⁵, or —S(C═O)CR⁴═CHR⁵; wherein R³, R⁴, and R⁵ are each independently H or —(C₁-C₆)alkyl; and each —(C₁-C₆)alkyl is independently unbranched or branched.
3. The activatable nanogel of embodiment 1 or 2 wherein L¹ and L² are —NH(C═O)CH═CH₂.
4. The activatable nanogel of any one of embodiments 1-3 wherein R¹ and R² are ethyl.
5. The activatable nanogel of any one of embodiments 1-4 wherein X¹ is chloro.
6. The activatable nanogel of any one of embodiments 1-5 wherein X⁻ is halo.
7. The activatable nanogel of embodiment 1 or 7 wherein the cyanine is:

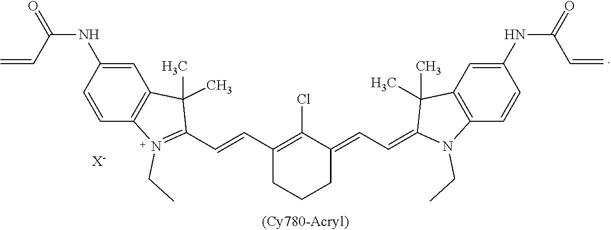

8. The activatable nanogel of any one of embodiments 1-7 wherein the monomer is N(R³)₂(C═O)CR⁴═CHR⁵, —O(C═O)CR⁴═CHR⁵, or —S(C═O)CR⁴═CHR⁵; wherein R³, R⁴, and R⁵ are each independently H or —(C₁-C₆)alkyl; and each —(C₁-C₆)alkyl is independently branched or unbranched.
9. The activatable nanogel of any one of embodiments 1-8 wherein the monomer is NH(CH(CH₃)₂)(C═O)CH═CH₂.
10. The activatable nanogel of any one of embodiments 1-9 wherein the crosslinked nanoparticle has a spherical morphology and an average diameter of about 80 nanometers to about 220 nanometers.

In some embodiments, the average diameter is about 50 nm, about 60 nm, about 70 nm, about 80 nm, about 90 nm, about 100 nm, about 110 nm, about 130 nm, about 140 nm, about 150 nm, about 160 nm, about 170 nm, about 180 nm, about 190 nm, about 200 nm, about 210 nm, about 220 nm, about 230 nm, about 240 nm, or about 250 nm.
11. The activatable nanogel of any one of embodiments 1-10 wherein the chemotherapeutic has a molecular weight of about 300 g mol⁻¹ to about 1500 g mol⁻¹.

In some embodiments, the chemotherapeutic has a molecular weight (in g mol⁻¹) of about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, or about 3000.

In various embodiments, the nanogel does not comprise molecular oxygen, ozone, peroxides, a free radical species, or a combination thereof.
12. The activatable nanogel of any one of embodiments 1-11 wherein the chemotherapeutic is paclitaxel, geftitinib, GW9662, or cisplatin.
13. A method for preparing an activatable nanogel according to any one of embodiments 1-12, which method comprises:
   a) forming an aqueous mixture comprising a surfactant, a compound of Formula I, and a monomer capable of crosslinking to L¹ and/or L² of Formula I;
   b) degassing the aqueous mixture;
   c) contacting the degassed aqueous mixture, a radical initiator, and a base to form a nanogel solution comprising a crosslinked nanoparticle;
   d) purifying the nanogel solution; and
   e) contacting the purified nanogel solution and a chemotherapeutic;
wherein the activatable nanogel is thereby formed.

In some embodiments, the aqueous mixture and/or the nanogel solution comprises an alcohol. In some embodiments, the alcohol is methanol or ethanol. In some embodiments, the surfactant is sodium dodecyl sulfate (SDS). In some embodiments, the radical initiator is ammonium persulfate. In some embodiments, the base is N,N,N,N-tetramethylethylenediamine. In some embodiments, the degassing is performed by bubbling an inert gas into to aqueous mixture. In some embodiments, forming of the nanogel solution is performed below ambient temperature, for example at 0 °C. In some embodiments, the purifying is performed by dialysis.
14. A method for treating cancer comprising:
   a) administering to a subject having a cancer an activatable nanogel according to any one of embodiments 1-12, wherein the cancer uptakes a crosslinked nanoparticle of the activatable nanogel;
   b) irradiating the cancer that has uptaken the crosslinked nanoparticle with near infrared light for a period of time sufficient to release a chemotherapeutic from the crosslinked nanoparticle of the activatable nanogel; wherein the cancer is thereby treated.

In some embodiments, the irradiating is at a wavelength of about 700 nm, about 720 nm, about 740 nm, about 760 nm, about 780 nm, about 800 nm, about 820 nm, about 840 nm, about 860 nm, about 880 nm, or about 900 nm. In various embodiments, irradiating for a period of time that is sufficient is about 5 minutes, about 10 minutes, about 15 minutes, about 30 minutes, about 45 minutes, about 60 minutes, about 75 minutes, about 90 minutes, about 120 minutes, about 150 minutes, about 180 minutes, or about 210 minutes. In various embodiments, the irradiating for a period of time that is sufficient is performed 2 or more time, wherein each period is separated by a pause in the irradiating that is equal in time to the period of time that is sufficient.
15. The method of embodiment 14 wherein the irradiating forms a 1,2-dioxetane at a carbon-carbon double bond of a cyanine of Formula I (see for example, Chart 1b), causing release of the chemotherapeutic from the crosslinked nanoparticle and into the cancer.

### Results and Discussion

### Design and Synthesis of Cy780-Acryl.

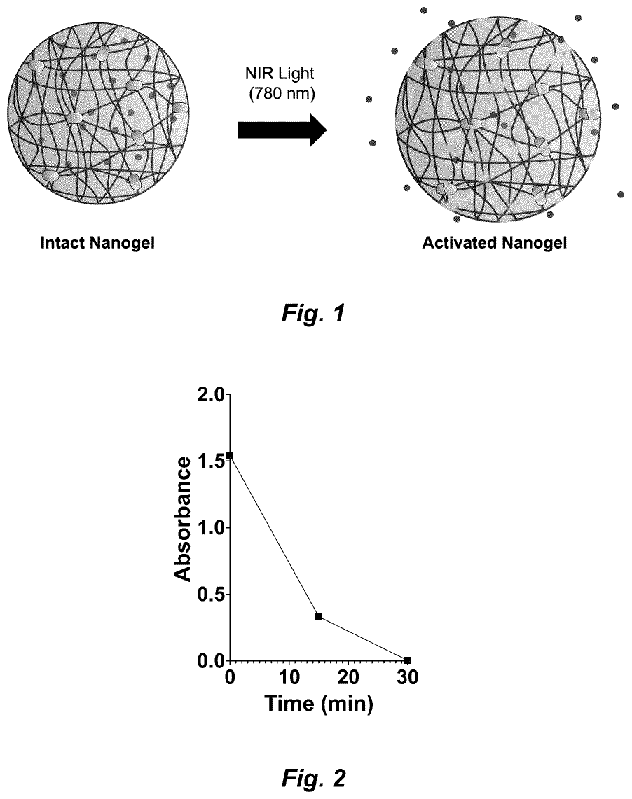

Our design utilizes acrylamide-based nanogels, which are nanoparticles comprised of a crosslinked hydrophilic network, as the cargo carrying system (Adv Drug Deliv Rev 2008, 60 (15), 1638-1649). In addition to crosslinking with N-isopropylacrylamide (NIPAM), we developed Cy780-Acryl and incorporated it into the hydrogel backbone (Chart 1a). Specifically, Cy780-Acryl is a modified version of Cy7-Cl which features two acrylamide units on the peripheral of the molecule. Of note, a cyclohexenyl fused cyanine was chosen to serve as the crosslinker backbone, as opposed to an ICG core, owing to its enhanced stability and ease of synthetic manipulation. We hypothesize that light irradiation at 780 nm (the λₘₐₓ) will generate an excited state that can interact with molecular oxygen to form an unstable dioxetane intermediate. Subsequent collapse of the dioxetane will yield two carbonyl fragments which facilitate separation within the polymer structure, nanogel degradation, and subsequent cargo release (Chart 1b and FIG. 1).

Chart 1. a) Chemical structures of NIPAM, Cy780-Acryl, and Cy780-Actamido. b) Spontaneous decomposition of unstable 1,2-dioxetane cyanine intermediates yields ketone and aldehyde fragments.

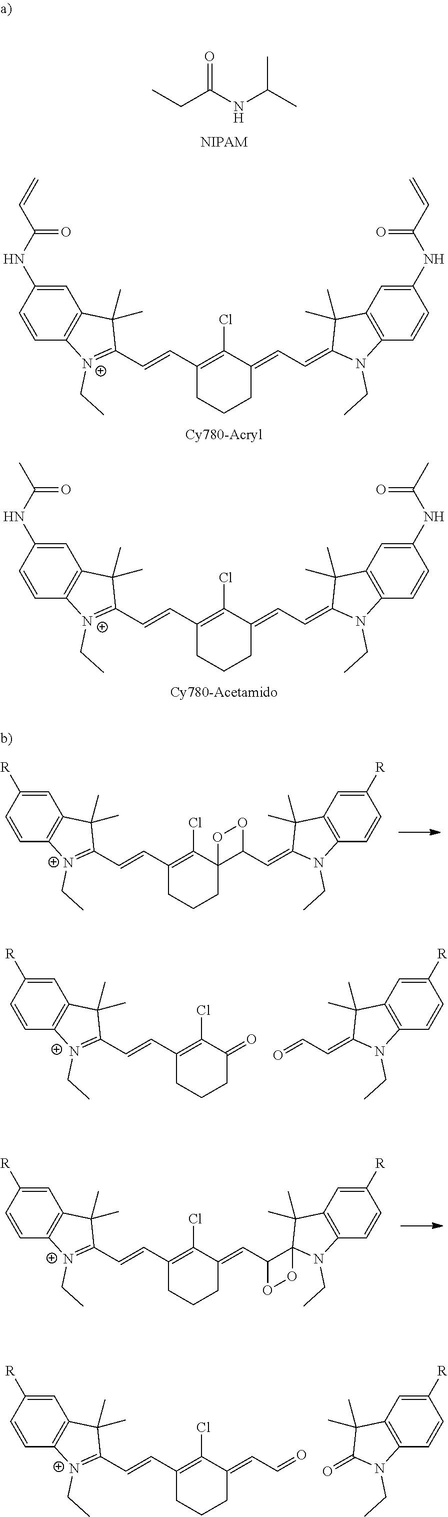

In brief, the synthesis of Cy780-Acryl began with the nitration of 2,3,3-trimethylindolenine using a sulfuric acid and nitric acid mixture to afford 1 in 95% yield. The nitro intermediate was then reduced in the presence of SnCl₂ and hydrochloric acid, followed by Boc protection using Boc₂O to furnish 3 in 41% yield over two-steps. Subsequently, 3 was alkylated with ethyl iodide to give 4 quantitatively. The Boc-protected cyanine 5 was obtained in 24% yield upon treatment of 3 with activated Schiff base 4 in the presence of acetic anhydride. Finally, the acrylamide units were appended by sequential Boc deprotection with TFA, followed by treatment with acryloyl chloride to afford Cy780-Acryl in 39% yield over two-steps (Scheme 1). In addition to Cy780-Acryl, we also synthesized Cy780-Acetamido, a control cyanine which is incapable of crosslinking to probe the proposed mechanism of NIR-light-mediated release (Chart 1a). Cy780-Acetamido was prepared in a similar manner using acetyl chloride in the final step (25% yield over two-steps).

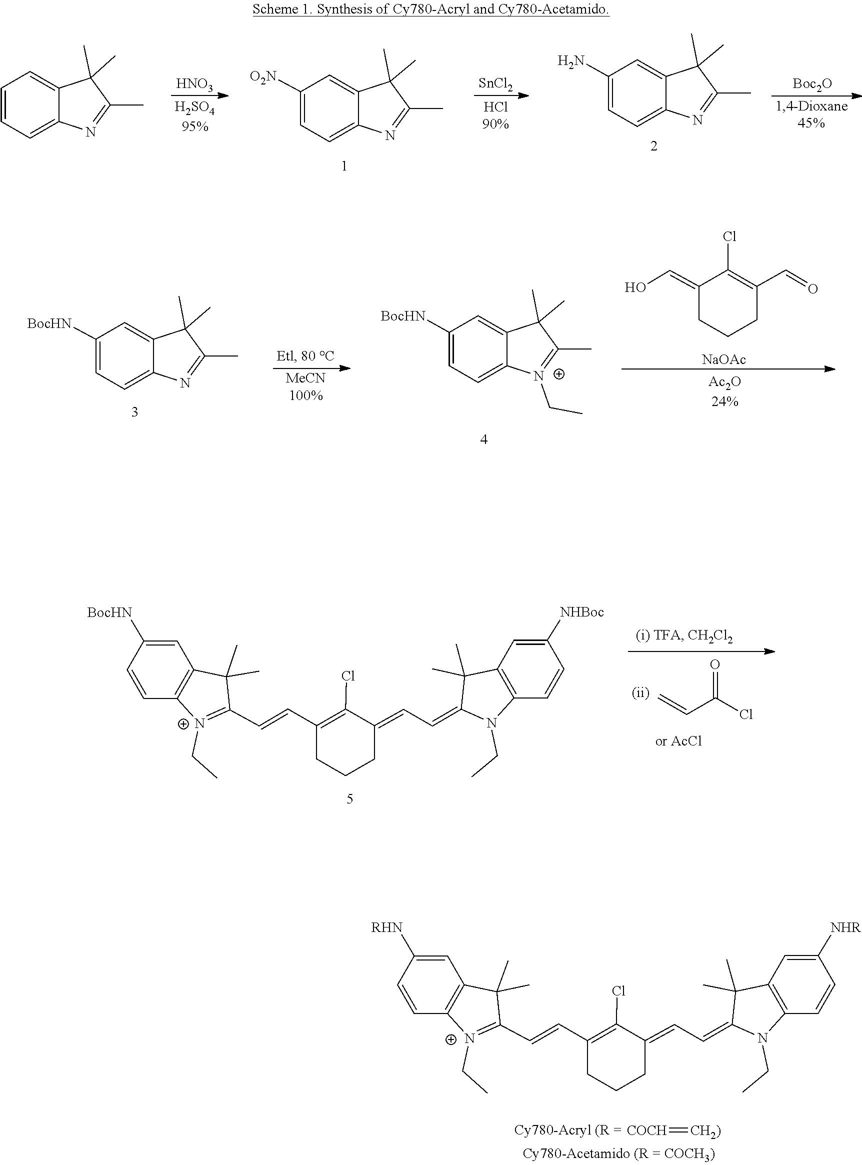

### Assessment of Photobleaching Properties.

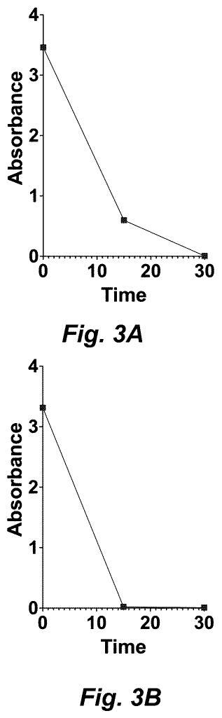

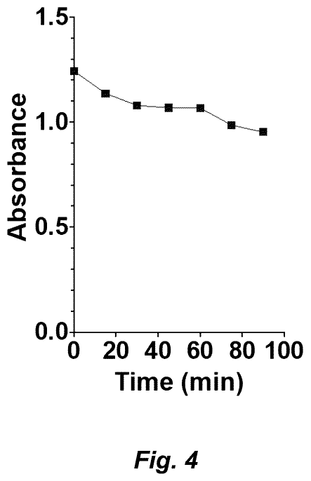

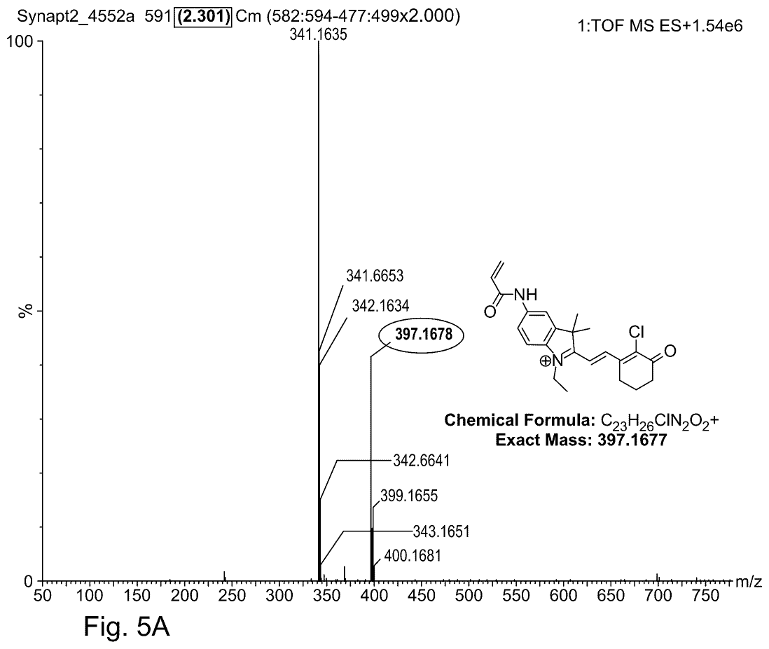

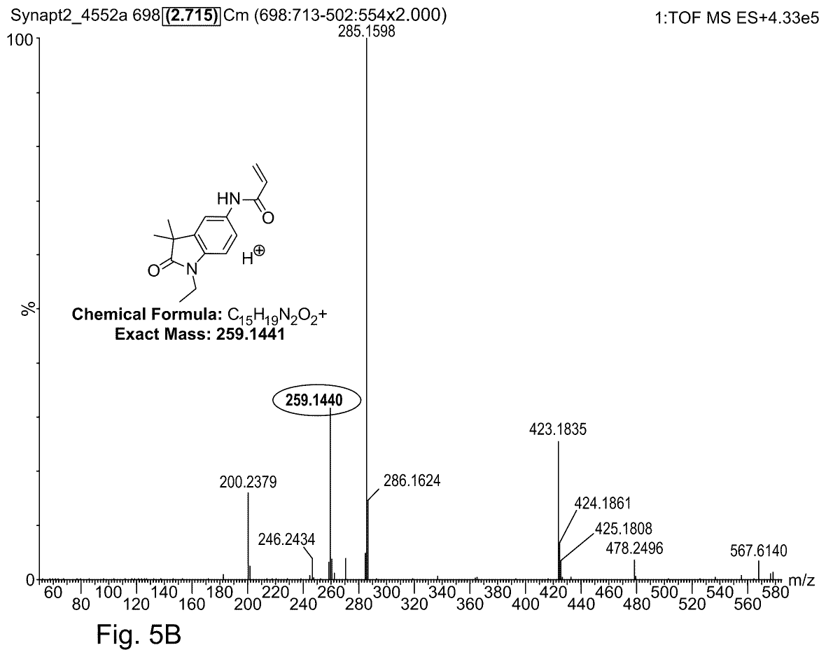

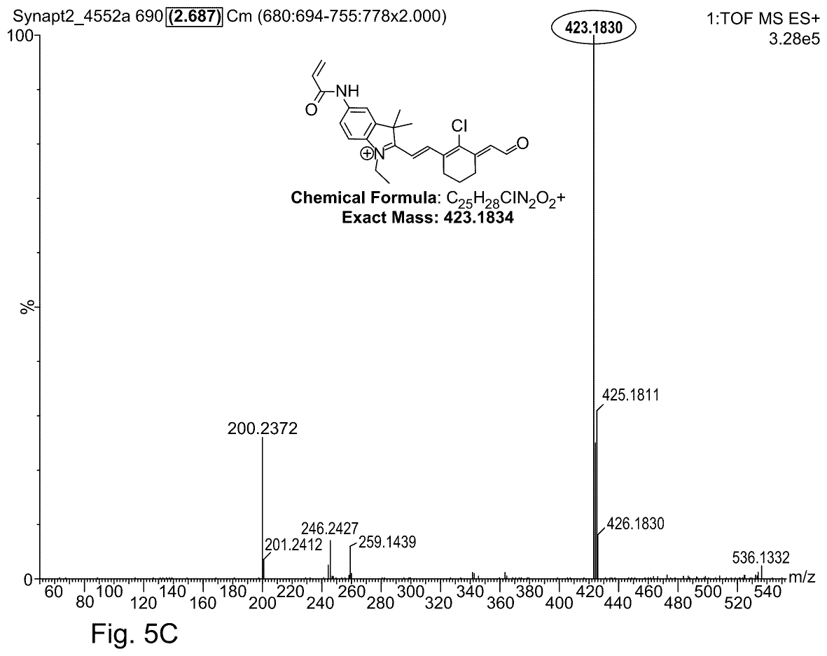

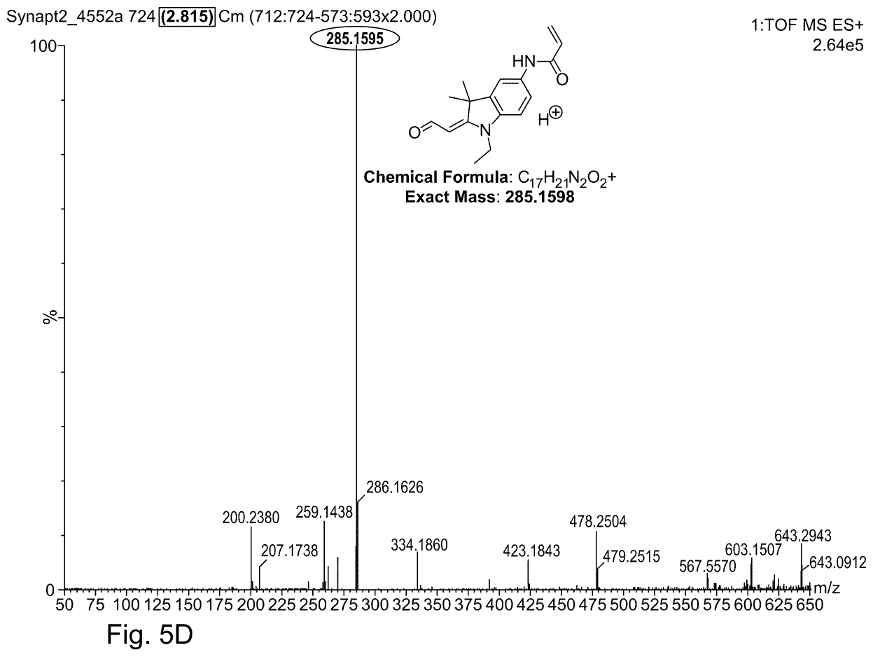

With the various components in hand, we turned our attention to testing whether the amide moiety would impact the photophysical properties of the cyanine core. First, we analyzed the absorption spectrum and measured the extinction coefficient of Cy780-Acryl. Compared to the Cy7-Cl reference in DMSO, the λₘₐₓ (831 nm cf. 793 nm) and extinction coefficients (1.11×10⁵ M⁻¹cm⁻¹ cf. 1.23×10⁵ M⁻¹cm⁻¹) were similar. Next, we evaluated the photostability of the crosslinker. When Cy780-Acryl (as well as Cy780-Acetamido) was irradiated with a LED light source at 780 nm (300 mW power output, 47.3 μW/mm² maximum irradiance), we found complete degradation of the molecule after only 25 minutes based on the disappearance of the NIR absorbance band (FIG. 2). This result is comparable to when Cy7-Cl is irradiated under identical conditions, demonstrating the chemical modifications we made did not significantly alter photobleaching properties (FIG. 3). Of note, when dissolved oxygen was removed prior to the initiation of photobleaching, we found the change in absorbance to be minimal, supporting the proposed dioxetane mechanism (FIG. 4). Indeed, the anticipated carbonyl fragmentation products were detected via LC-MS when samples were irradiated in the presence of oxygen (FIG. 5).

### Fabrication of NIR-Nanogels.

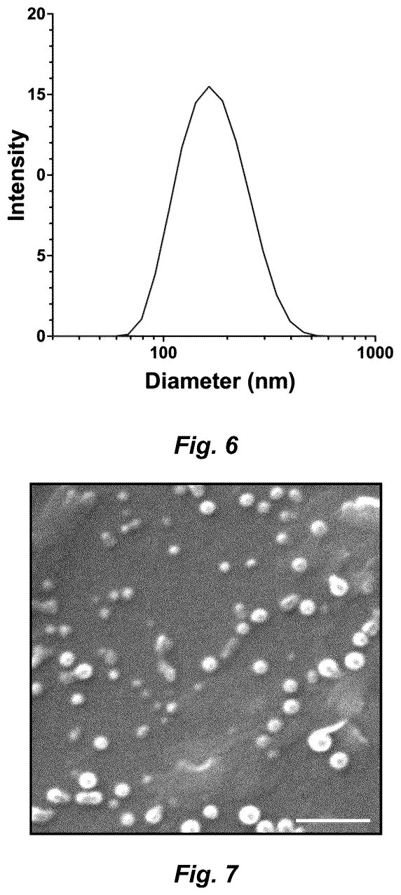

Next, we turned our attention to fabricating NIR-nanogels with the following key properties in mind. First, we targeted a size distribution between 100-200 nm to ensure efficient tumor uptake. Second, NIR-light irradiation should be the only input that triggers cargo release. Finally, we aimed to develop a robust delivery system that exhibits minimal off-target toxicity prior to light activation. The synthesis of the NIR-nanogels was accomplished through a self-assembly method using reverse micelles in aqueous solution (J Am Chem Soc 2010, 132 (39), 13648-13650). We selected this approach because the size distribution can be controlled by varying the concentration of each polymeric component, as well as the micelle composition. In brief, the NIPAM monomer and the Cy780-Acryl crosslinker were rapidly stirred in Milli-Q water in the presence of SDS at a molar ratio of 0.98 and 0.02, respectively. After the removal of dissolved oxygen from the solution, polymerization was initiated by adding ammonium persulfate (APS) and TEMED. The resulting nanogels were then purified via dialysis and analyzed using dynamic light scattering (DLS) measurements. We found an average hydrodynamic diameter of 125 nm±41 nm, which was within our target size range and a polydispersity index (PDI) of 0.15, indicating a normal size distribution (FIG. 6). To support these results, we employed scanning electron microscopy (SEM) and obtained a size distribution of 110 nm±35 nm (FIG. 7). It is noteworthy that the particles exhibited a spherical morphology, which is consistent with other nanogel systems.

### In Vitro Evaluation of NIR-Nanogel.

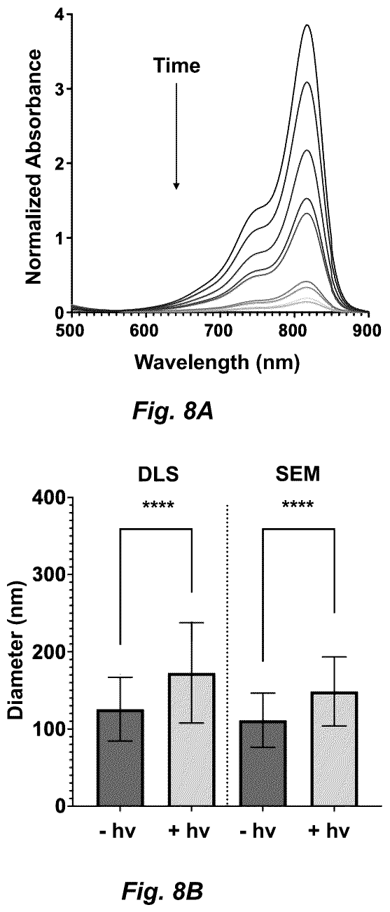

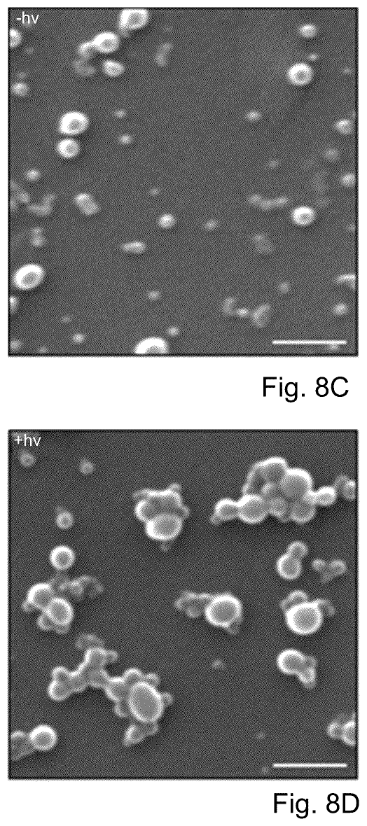

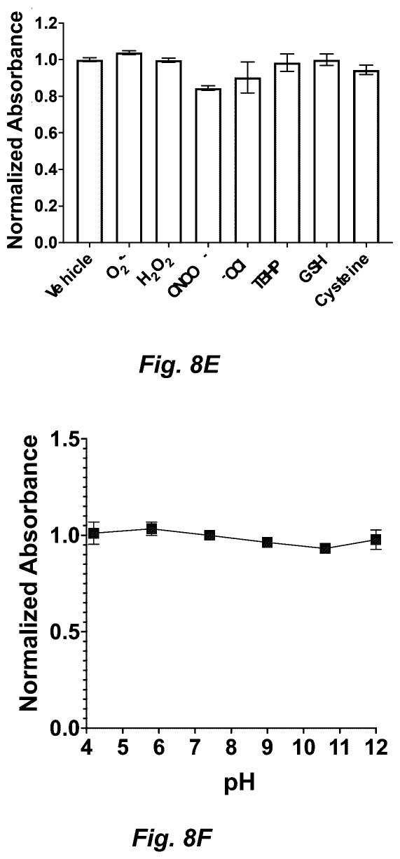

To assess responsiveness to NIR light, the particles we obtained were irradiated at 780 nm. Although the loss of NIR absorbance was slower than the free dye, the requisite photobleaching was observed (FIG. 8a). DLS analysis of the dark sample showed there was no change in size (FIG. 8 2b) and further, we found the particles were stable for up to three months. In contrast, the NIR light-treated samples were degraded after 30 minutes and showed a size increase of 47 nm by DLS (FIG. 8b). Moreover, we observed a significant change in morphology from spheres to an irregular appearance, as well as clear signs of coalescence (FIG. 8c and FIG. 8d). In comparison, hydrogels synthesized with the control non-crosslinker (Cy780-Acetamido), displayed a size distribution of 120 nm by DLS, with no observable absorbance coming from the cyanine after dialysis purification. As anticipated, exposure to NIR-light did not result in a change of the particle size, highlighting the critical role played by the Cy780-Acryl crosslinker.

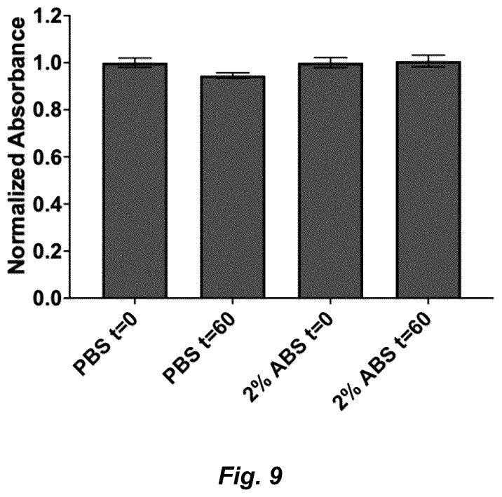

Furthermore, NIR-nanogels were treated with a panel of biologically relevant analytes to assess potential issues with stability when applied in vivo. Reactive oxygen and nitrogen species (O₂*⁻, H₂O₂, ·OH, ONOO⁻, ClO⁻), and biological thiols (cysteine and glutathione) were amongst those tested. Our results indicate that in each instance, our nanogel system exhibited good stability, presumably due to the hydrogel network that can protect Cy780-Acryl from decomposition (FIG. 8e). For instance, although ONOO⁻ and ClO⁻ can rapidly decompose various cyanine dyes in free solution, we only observed a 16% and 10% decrease in absorbance, respectively even at high concentrations (0.1 mM). We also assessed nanogel stability across a pH range spanning from 4.2 to 12.0. Our findings indicate the NIR-nanogels are resistant to changes in pH (FIG. 8f). Stability in the presence of serum was also evaluated owing to possible interactions in vivo; however, we did not find any effect on Cy780-Acryl absorbance upon co-incubation (FIG. 9).

### Cargo Selection, Encapsulation, and Light-Mediated Release.

After establishing a robust protocol to fabricate NIR-nanogels displaying the desired properties, we turned our attention to identifying cargo to showcase its utility for in vivo applications such as the treatment of breast tumors. Cisplatin, gefitinib, and GW9662 were identified as candidates owing to the absence of a convenient covalent attachment point (e.g., carboxylate, hydroxyl) (Chart 2). In the context of mode of action, cisplatin was selected because it can trigger apoptosis of breast cancer cells (as well as other cancers) through binding to DNA base pairs to yield DNA adducts. Likewise, gefitinib is an epidermal growth factor receptor (EGFR) inhibitor based on an anilino quinazoline scaffold used to treat certain breast cancer types. Lastly, GW9662 is an N-phenylbenzamide analog that can selectively and irreversibly inhibit unregulated peroxisome proliferator activated receptor-γ (PPARγ) found in breast cancer. It is noteworthy we also selected paclitaxel for encapsulation due to its ubiquitous use in breast cancer treatment.

Chart 2. Chemical structures of encapsulation cargo employed in this study.

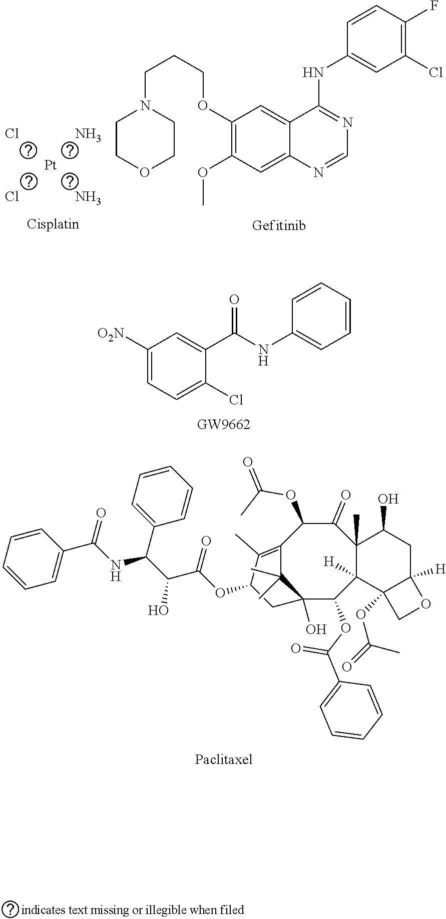

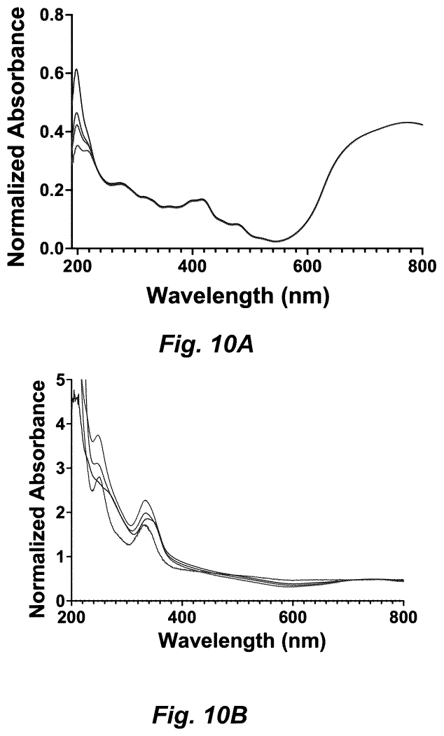

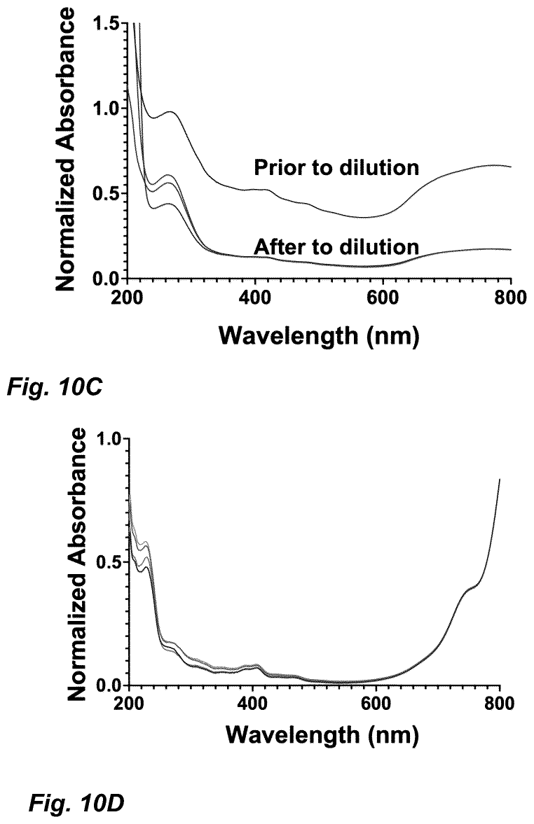

Cargo encapsulation using standard sonication protocols proved to be challenging in our hands as we were unable to achieve consistent loading results. After extensive screening, we obtained an optimized procedure that yielded reliable encapsulation results for each drug. We discovered that slow addition (versus bolus addition) of the drug to NIR-nanogel samples was essential to maintain a homogeneous suspension of the particles in solution. Further, the inclusion of at least 10% ethanol as a solubilizing co-solvent was necessary for drug encapsulation. However, beyond 25% ethanol content, we observed swelling of the hydrogels, leading to an increased hydrodynamic diameter above our targeted size distribution of 100-200 nm. After removal of non-encapsulated drug, we established a method to assess passive leeching of the contents by dialyzing the drug-loaded sample against MilliQ water. Small aliquots were removed at various time points and compared to the initial cargo concentration. We found that cisplatin displayed the highest degree of leeching (~50%), while gefitinib showed a concentration loss of ~6% after three hours of dialysis. On the contrary, no passive release of GW9662 was observed. The corresponding data for paclitaxel also demonstrates minimal leeching (FIG. 10). In general, the extent of passive cargo loss was determined by placing a photobleached sample of each drug-loaded NIR-nanogel in 3 kDa MWCO dialysis tubing for dialysis against DI water. Aliquots were taken to determine the absorbance at various time points for comparison against the corresponding calibration curve.

### Drug Delivery in a Murine Model of Breast Cancer.

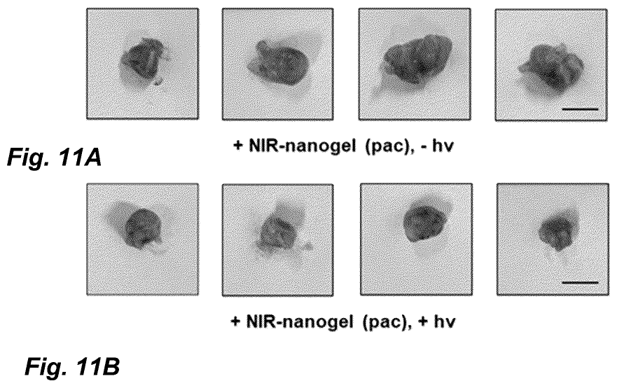

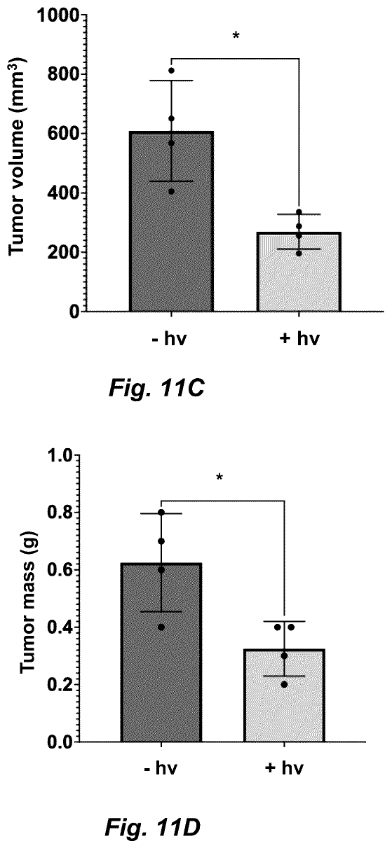

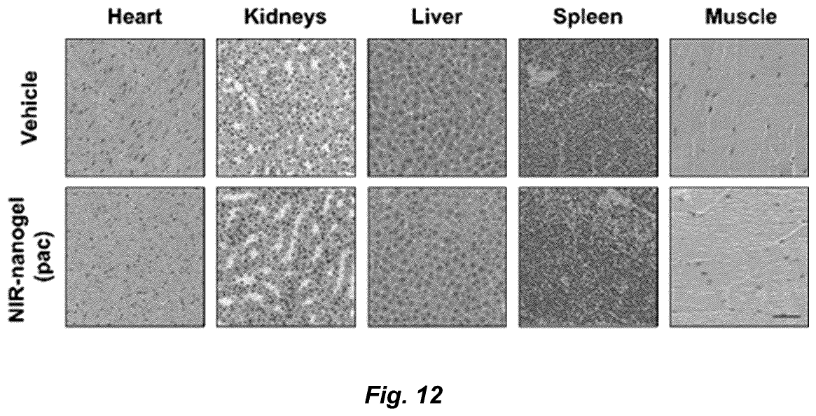

Next, a murine model of breast cancer was established by inoculating BALB/c mice (6-8 weeks old) with 4T1 cells. The animals were then randomly divided into two groups. Once the tumors grew to ~150 mm³ in volume, all animals were treated with paclitaxel-loaded nanogels (NIR-nanogel (Pac)) at a dose of 2 mg/kg via systemic administration. After 30 minutes, the experimental group of animals were treated with NIR light from a 780 nm LED positioned 2 cm away from the tumor site. Of note, the total irradiation duration was 30 minutes, which was separated into two 15-minute sessions with a 15-minute interval. Since oxygen is required to fragment Cy780-Acryl, we hypothesized this sequence would be critical to ensure that the tumor adequately reoxygenates. Each mouse was treated every other day for a total of three treatments. Compared to the control group of animals that did not receive light, the tumors were 44% smaller as determined by the caliper method. We confirmed these results by excising the tumors after the animals were sacrificed (FIG. 11a-d). To verify that the nanogel delivery system can reduce the off-target toxicity that is typically associated with systemic administration of a drug such as paclitaxel, we harvested the vital organs (heart, kidneys, liver, muscle, and spleen) from a cohort of mice were administered paclitaxel-loaded NIR-nanogels to perform H&E staining (FIG. 12).

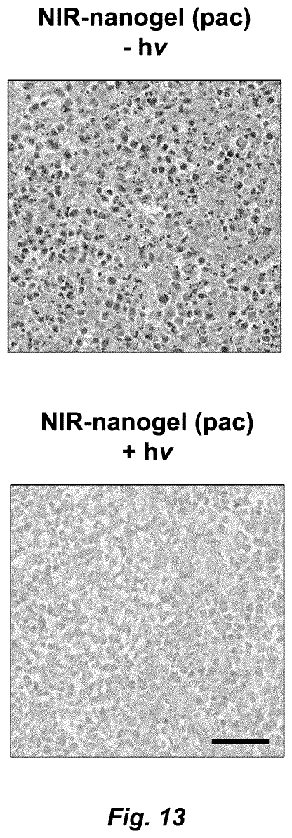

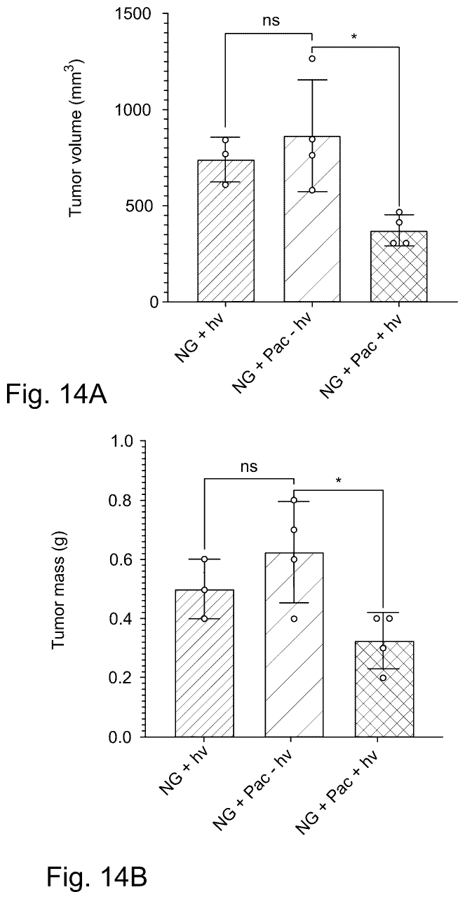

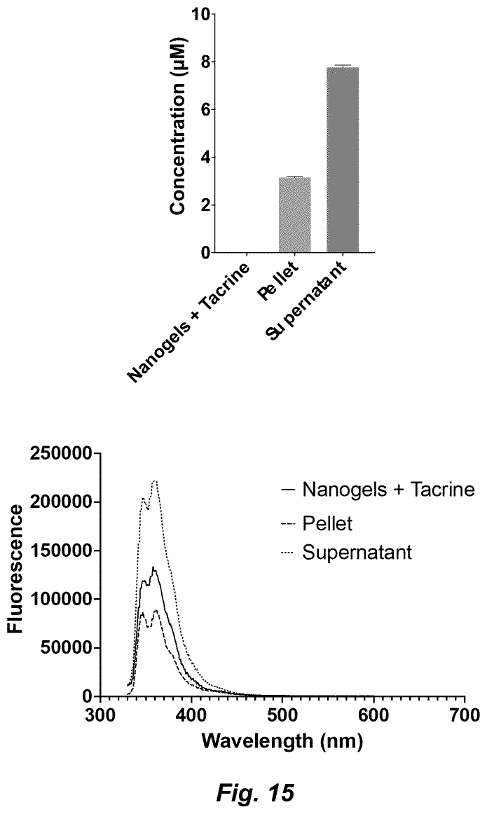

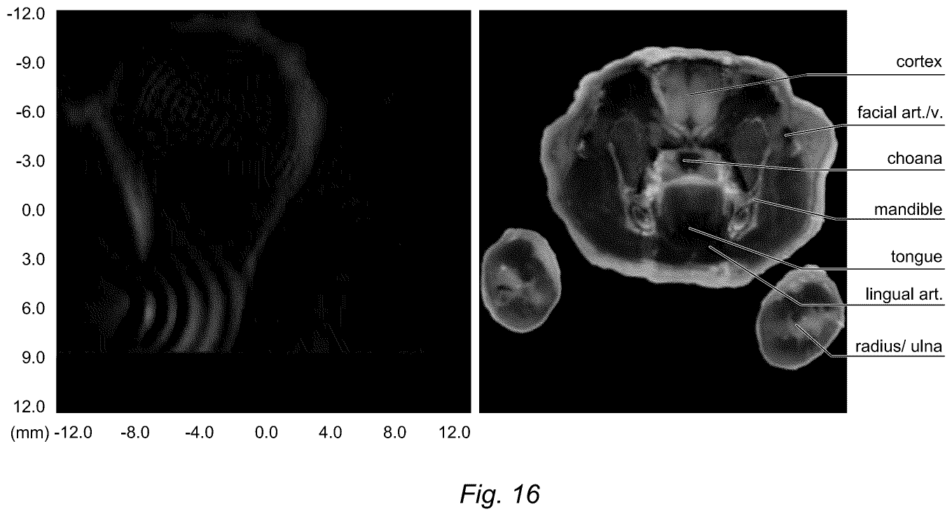

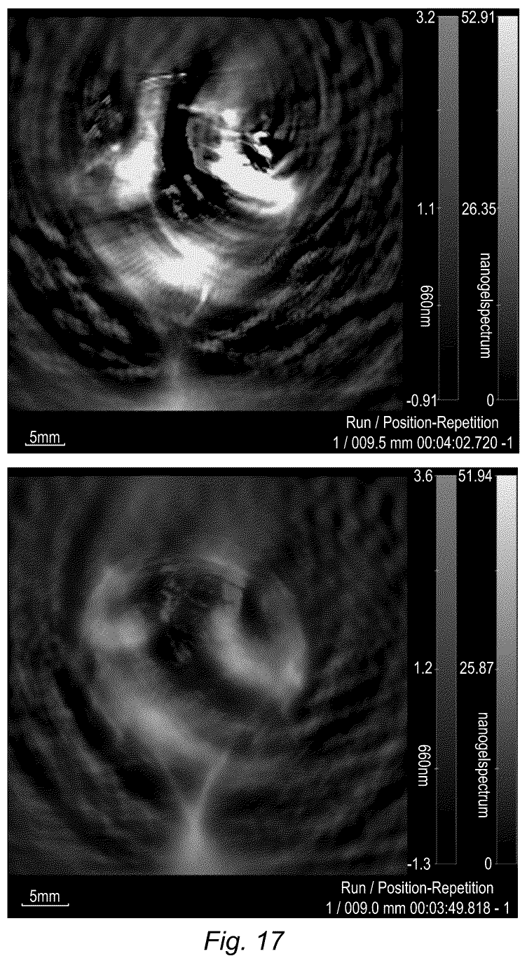

Analysis of the histological data revealed no discernable difference relative to control mice. This is in contrary to histology performed on excised tumors from animals treated with paclitaxel-loaded NIR-nanogels, followed by either light irradiation or no light. Our results show massive differences in nuclear staining pattern between the two groups, indicating cell death results from drug release (FIG. 13). Finally, we performed a series of experiments to determine whether attenuation of tumor growth involved singlet oxygen-mediated cell death. First, we used esterase-activated 2′,7′-dichlorodihydrofluorescein diacetate (DCF-DA) to detect ROS (including singlet oxygen) from a sample of light-irradiated NIR-nanogel. We found that upon irradiation, the signal from the DCF scavenger increased by four-fold. Next, we performed a complementary animal experiment to determine if the in vitro result translates into an in vivo setting. Tumors were treated with unloaded NIR-nanogels and then dosed with light. We hypothesized that if singlet oxygen production in vivo contributed to the observed attenuation of tumor growth, irradiation of nanogels without paclitaxel would result in smaller tumors relative to the dark controls. As shown in FIG. 14, no statistical difference based on tumor volume or tumor mass measurements were noted. The discrepancy between the in vitro and in vivo experiments is likely due to a lower effective singlet oxygen concentration within the tumor.

Conclusion. In closing, we have developed the first NIR light-activatable nanogel delivery system that utilizes cyanine photobleaching chemistry. Light, especially at wavelengths within the NIR window (650 to 900 nm), is one of the most powerful triggers because it does not generally harm tissue (minimal phototoxicity) and can penetrate deep within the body because there are fewer endogenous chromophores present to absorb or scatter incident light. Moreover, light can be focused onto a region of interest to afford remarkable spatiotemporal control. This is especially the case in the context of fluorescent- and photoacoustic-based imaging studies, optogenetic stimulation, photodynamic therapy (PDT) or photothermal therapy (PTT), and light-activatable analyte donor systems (J Am Chem Soc 2020, 142 (2), 680-684). With regards to the latter two applications, we envision existing laser and LED systems used in humans can also be compatible with the NIR-nanogel technology, highlighting its translational potential. As shown in the current work, NIR-nanogels can encapsulate a diverse range of cargo, including chemotherapeutics that do not contain a site for covalent modification. The ability to keep a drug encased while the NIR-nanogels are in systemic circulation spares the rest of the body from undesirable off-target toxicity and offers an exciting complimentary approach to conventional prodrug design strategies. Through targeted irradiation, we were able to significantly attenuate tumor growth (via local paclitaxel release), while avoiding collateral damage of healthy tissue as demonstrated by histological staining experiments. Despite these excellent in vivo results obtained from this pilot study, we anticipate further tuning of the amount of cargo loaded, as well as optimization of the precise time and duration of light treatment will improve NIR-nanogel performance. In addition to such efforts, which are currently underway, we are also exploring the encapsulation of other cargo for a diverse range of biomedical applications.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, a-ketoglutarate, and P-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

### Materials:

Materials were purchased from commercial vendors and used without further purification. All deuterated solvents were purchased from Cambridge Isotope Laboratories. Acetone, dichloromethane, phosphate saline buffer (Corning), potassium iodide, sodium bicarbonate, sodium carbonate and sodium hydroxide were purchased from Thermo Fisher Scientific. Anhydrous methanol was purchased from Macron Fine Chemicals. Concn. hydrochloric acid was purchased from VWR BDH Chemicals. Anhydrous acetonitrile, anhydrous dichloromethane, anhydrous dimethylformamide, sodium hydride, acryloyl chloride, celite 545, and Hexanes were purchased from Millipore-Sigma Aldrich. Sodium sulfate (anhydrous) was purchased from Oakwood Chemicals. 4-Aminophenol was purchased from AK Scientific. Chloroform was purchased from OmniSolv.

### Instruments and Software:

¹H and ¹³C NMR spectra were acquired on a Varian 500 or Carver B500 spectrometer. The following abbreviations were used to describe coupling constants: singlet (s), doublet (d), triplet (t), quartet (q) or multiplet (m). Spectra were visualized and analyzed using MestReNova (version 14.2) and referenced to trace non-deuterated solvent. High-resolution mass spectra were acquired on a Waters Q-TOF Ultima ESI mass spectrometer or a Waters Synapt G2-Si ESI/LC-MS spectrometer. Data was analyzed using Microsoft Excel and GraphPad Prism (version 6.0 or 8.0) and final figures were prepared in PowerPoint (version Microsoft 365). Absorption spectra was acquired on a JASO V-770 UV-Vis/NIR spectrophotometer for wavelengths ranging from 190-1200 nm. DLS measurements were acquired with a Malvern Zetasizer Nano ZS.

### Experimental Procedure:

All procedures to fabricate and purify NIR-nanogels were performed using Milli-Q water unless otherwise specified.

### Example 2. Synthetic Procedures

Thin-layer chromatography (TLC) was performed on glass-backed TLC plates precoated with silica gel containing an UV254 fluorescent indicator (Macherey-Nagel). TLCs were visualized with a 254/365 nm UV hand-held lamp (UVP). Flash silica gel chromatography was performed using 0.04-0.063 mm 60 M silica (Macherey-Nagel). All glassware used under anhydrous reaction conditions were flame-dried under vacuum and cooled immediately before use.

### 2,3,3-Trimethyl-5-nitro-3H-indole (Compound 1)

2,3,3-Trimethylindolenine (1.0 g, 6.3 mmol, 1 eq) was cooled to 0 °C and mixed with sodium nitrate (0.59 g, 6.9 mmol, 1.1 eq). While stirring, concentrated sulfuric acid (16.0 mL, 300 mmol, 48 eq) was added dropwise over 10 minutes. The solution was stirred at the same temperature for 1 hour. The mixture was then neutralized with solid NaOH at 0 °C. The material was then dissolved with EtOAc and washed 3× with water (20 mL). The organics were collected, dried over sodium sulfate, filtered, and concentrated under reduced pressure resulting in 1.2 g of the titled compound as a red/orange solid in 95% yield, which was used without further purification. 1H NMR (500 MHz, CDCl3) δ 8.26 (dd, J=8.5, 2.3 Hz, 1H), 8.16 (d, J=2.3 Hz, 1H), 7.61 (d, J=8.5 Hz, 1H), 2.35 (s, 3H), 1.37 (s, 6H). 13C NMR (125 MHz, CDCl3) δ 16.11, 22.88, 54.65, 117.31, 120.20, 124.70, 145.81, 146.83, 159.09, 194.27.

### 2,3,3-Trimethyl-3H-indol-5-amine (Compound 2)

Compound 1 (1.0 g, 4.9 mmol, 1 eq) was suspended in a 6 M HCl solution (27 mL). Tin(II) chloride dihydrate (6.1 g, 27 mmol, 5.5 eq) was then added to the reaction and heated and stirred at 100 °C for 2 hours. The mixture was cooled to room temperature and then neutralized with aqueous NaOH. The material was extracted 3× with EtOAc (20 mL), the organics were collected, dried over sodium sulfate, filtered, and concentrated under reduced pressure. The crude material was then purified via silica flash column chromatography using a gradient of 0-100% EtOAc/Hexanes (v/v) resulting in 0.77 g of the titled compound as a yellow solid in 90% yield. 1H NMR (500 MHz, CDCl3) δ 7.29 (d, J=8.1 Hz, 1H), 6.61 (d, J=2.3 Hz, 1H), 6.59-6.56 (m, 1H), 3.47 (s, 2H), 2.20 (s, 3H), 1.24 (s, 6H). 13C NMR (125 MHz, CDCl3) δ 15.25, 23.36, 53.47, 109.02, 113.95, 120.26, 144.38, 146.09, 147.39, 184.27.

### Tert-butyl (2,3,3-trimethyl-3H-Indol-5-yl)carbamate (Compound 3)

Compound 2 (750 mg, 4.3 mmol, 1 eq) was dissolved in 1,4-dioxane (5.2 mL, 60.3 mmol). Di-tert-butyl dicarbonate (1.13 g, 5.2 mmol, 1.2 eq) was added. The reaction was heated and stirred at 100 °C for 2.5 hours. The reaction was cooled to room temperature and the volatiles were removed under reduced pressure. The resulting crude material was then purified via silica flash column chromatography using a 1:1 EtOAc/Hexanes (v/v) resulting in 936 mg of the titled compound as an orange solid in 79% yield. 1H NMR (500 MHz, CDCl3) δ 7.61 (s, 1H), 7.41 (d, J=8.2 Hz, 1H), 7.00 (dd, J=8.2, 2.3 Hz, 1H), 6.54 (s, 1H), 2.25 (s, 3H), 1.52 (s, 9H), 1.29 (s, 6H). 13C NMR (125 MHz, CDCl3) δ 15.49, 23.24, 28.52, 54.05, 80.62, 112.58, 117.92, 119.93, 136.07, 146.86, 153.01, 187.20.

### 5-((tert-Butoxycarbonyl)amino)-1-ethyl-2,3,3-trimethyl-3H-indol-1-ium (Compound 4)

Compound 3 (830 mg, 3.03 mmol, 1 eq) was dissolved in MeCN (15 mL). Iodoethane (1.22 mL, 15.1 mmol, 5 eq) was added and the reaction was refluxed at 85 °C for 12 hours. The reaction was cooled to room temperature, dissolved with minimal amount of acetone and slowly dropped into a flask containing chilled diethyl ether. The resulting precipitate that formed was collected via vacuum filtration and dried under vacuum to obtain compound 4 in quantitative yields, which was used without further purification. 1H NMR (500 MHz, CDCl3) δ 8.01 (d, J=5.9 Hz, 1H), 7.84 (s, 1H), 7.58 (d, J=8.6 Hz, 1H), 4.55 (q, 2H), 2.94 (dd, J=5.5, 2.4 Hz, 3H), 2.16-2.08 (m, 2H), 1.52 (s, 9H), 1.48-1.41 (m, 9H). 13C NMR (125 MHz, CDCl3) δ 13.86, 16.54, 23.24, 23.34, 28.47, 28.58, 31.24, 45.42, 54.69, 113.48, 113.55, 115.75, 119.33, 135.05, 141.60, 142.98, 153.01.

### Cy780-Acryl.

Synthesis of Cy780-Acryl was performed over 3-steps. First, compound 3 (1.0 g, 2.955 mmol, 2 eq) was mixed with (E)-2-chloro-3-(hydroxymethylene)cyclohex-1-ene-1-carbaldehyde (255 mg, 1.477 mmol, 1 eq). The solids were then dissolved in acetic anhydride (5 mL) under a nitrogen atmosphere. Sodium acetate (242 mg, 2.96 mmol, 2 eq) was added and the reaction was heated to 130 °C for one hour. The reaction was allowed to cool to room temperature and the resulting precipitate that formed was collected via vacuum filtration and washed sequentially with diethyl ether and a aqueous solution of potassium iodide. The solid was collected and dried under vacuum and further purified via silica gel flash column chromatography using 7:93 MeOH/CH₂Cl₂ (v/v). The intermediate (105 mg) was dissolved in CH₂Cl₂ (515 μL) and treated with 2,2,2-trifluoroacetic acid (197 μL, 18 eq) and stirred at room temperature for 12 hours. The reaction was neutralized using a saturated aqueous solution of sodium bicarbonate and extracted with CH₂Cl₂.

The organic fractions were combined and washed with a solution of potassium iodide. The organic layer was collected, dried over sodium sulfate, and concentrated under reduced pressure. The material was used without further purification. This material (67 mg) was dissolved in MeCN (1.25 mL) under a nitrogen atmosphere, cooled to 0 °C and treated with sodium carbonate (65.8 mg, 621 μmol, 5 eq). A solution of acryloyl chloride (37.15 μL, 457 μmol, 3.7 eq) in MeCN (100 L) was added to the reaction dropwise. The reaction was allowed to warm to room temperature, then potassium iodide was added (246 mg, 1.48 mmol, 12 eq). The mixture was allowed to stir for 12 hours. The crude material was diluted with CH₂Cl₂ and washed with brine. The organic layer was dried over sodium sulfate, filtered, and concentrated under reduced pressure. The crude product was then purified via silica gel flash column chromatography initially using 1:19 MeOH/CH₂Cl₂ (v/v), and then 3:17 MeOH/CH₂Cl₂ (v/v) to give Cy780-Acryl (19 mg) as a green metallic solid in 25% yield. ¹H NMR (500 MHz, CD₃OD) δ 8.42 (d, J=14.1 Hz, 2H), 7.96 (d, J=1.9 Hz, 2H), 7.61 (dd, J=8.6, 1.9 Hz, 2H), 7.32 (d, J=8.6 Hz, 2H), 6.50-6.36 (m, 4H), 6.28 (d, J=14.1 Hz, 2H), 5.81 (dd, J=9.6, 2.2 Hz, 2H), 4.21 (q, J=7.2 Hz, 4H) 2.74 (t, J=6.0 Hz, 4H), 1.97 (p, J=6.1 Hz, 2H), 1.74 (s, 12H), 1.42 (t, J=7.2 Hz, 6H). ¹³C NMR (125 MHz, CD₃OD) δ 173.10, 166.03, 150.63, 144.93, 143.50, 139.46, 137.86, 132.27, 128.12, 127.90, 121.64, 115.83, 112.27, 102.07, 71.53, 50.73, 40.44, 28.27, 27.34, 22.16, 12.51.

### Cy780-Acetamido.

Synthesis of Cy780-Acetamido was performed over 3-steps. First, compound 3 (1.0 g, 2.955 mmol, 2 eq) was mixed with (E)-2-chloro-3-(hydroxymethylene)cyclohex-1-ene-1-carbaldehyde (255 mg, 1.477 mmol, 1 eq). The solids were then dissolved in acetic anhydride (5 mL) under a nitrogen atmosphere. Sodium acetate (242 mg, 2.96 mmol, 2 eq) was added and the reaction was heated to 130 °C for one hour. The reaction was allowed to cool to room temperature and the resulting precipitate that formed was collected via vacuum filtration and washed sequentially with diethyl ether and an aqueous solution of potassium iodide. The solid was collected and dried under vacuum and further purified via silica gel flash column chromatography using 7:93 MeOH/CH₂Cl₂ (v/v). The intermediate (105 mg) was dissolved in CH₂Cl₂ (515 μL) and treated with 2,2,2-trifluoroacetic acid (197 μL, 18 eq) and stirred at room temperature for 12 hours. The reaction was neutralized using a saturated aqueous solution of sodium bicarbonate and extracted with CH₂Cl₂.

The organic fractions were combined and washed with a solution of potassium iodide. The organic layer was collected, dried over sodium sulfate, and concentrated under reduced pressure. The material was used without further purification. This material (67 mg) was dissolved in MeCN (1.25 mL) under a nitrogen atmosphere, cooled to 0 °C and treated with sodium carbonate (65.8 mg, 621 μmol, 5 eq). A solution of acetyl chloride (32.7 μL, 459 μmol, 3.7 eq) in MeCN (100 L) was added to the reaction dropwise. The reaction was allowed to warm to room temperature, then potassium iodide was added (246 mg, 1.48 mmol, 12 eq). The mixture was allowed to stir for 12 hours. The crude material was diluted with CH₂Cl₂ and washed with brine. The organic layer was dried over sodium sulfate, filtered, and concentrated under reduced pressure. The crude product was then purified via silica gel flash column chromatography initially using 1:19 MeOH/CH₂Cl₂ (v/v), and then 3:17 MeOH/CH₂Cl₂ (v/v) to give Cy780-Acryl (19 mg) as a green metallic solid in 24% yield.

### Example 3. Preparation of Nanogels, Methods of Treatment and Assessment

### NIR-Nanogel Synthesis:

NIR-nanogels were prepared by mixing 200 μL of a 20 mg/mL SDS solution in a 50 mL round-bottom flask equipped with a magnetic stir bar. Subsequently, 1.44 mL of a 10 mg/mL NIPAM solution was added followed by 17.76 mL of Milli-Q water. The flask was placed on an ice-bath and the mixture was stirred on a magnetic stir plate (500 rpm). Cy780-Acryl (1.6 mg) was then dissolved in MeOH (600 μL) and this was added to the reaction vessel resulting in a total combined concentration of 6.5 mM of monomer and crosslinker. The mixture was allowed to stir at the same temperature while dry nitrogen gas bubbled through the mixture. After 30 minutes, 46 μL of a 60 mg/mL solution of APS was added slowly, followed by 44 μL of a 10% v/v TEMED solution in Milli-Q water. The mixture was allowed to stir at 0 °C and slowly warmed to room temperature for at least 12 hours. The sample was dialyzed using a 3.5 kDa MWCO dialysis tubing against DI water for two days with water changes twice a day.

### Paclitaxel Loading.

To encapsulate Paclitaxel, 4.5 mL of the above nanogel solution was placed in a 20 mL vial equipped with a magnetic stir bar. EtOH (71 μL) was then added, and the solution was stirred at 600 rpm on a magnetic stir plate. A 1.75 mM solution of Paclitaxel in EtOH (429 μL) was added dropwise while stirring. The solution was allowed to stir overnight in the dark. Excess drug was removed by centrifuging the nanogels at 10,000×g for 20 minutes, removing the supernatant and washing the nanogels with DI water 3x and finally resuspending in PBS buffer for next experiments.

Drug loading for other molecules followed the same general procedure to yield a final concentration of 200 M.

### In Vivo Nanogel Treatment:

BALB/c mice (6-8 weeks old) were inoculated with 4T1 cells (50 μL of 1×10⁶ cells/mL in 1:1 serum-free RPMI 1640 media and Matrigel (v/v)) via subcutaneous injection into one of the flanks. Once tumor volume reached 150 mm³ as determined via the caliper method, mice were treated with drug-encapsulated nanogels with light irradiation, drug-encapsulated nanogels without light irradiation, or no-drug nanogels with light irradiation. The general workflow for treatment consists of intravenous injection of nanogels, followed by a circulation period for 10-minutes, 15-minute irradiation, 10-minute rest, and finally another 15-minute irradiation. Irradiation was done with a Thorlabs, Inc.© 780 nm LED (300 mW power output, 47.3 μW/mm² maximum irradiance) at a distance of 1.0 cm away from the tumor area. Mice in each experimental group was treated for a total of 3 times on days 1, 3, and 5 and monitored for tumor volume and weight every day up to day 7. Subsequently, mice were euthanized, and tissue samples were harvested. Mice were irradiated with a 780 nm LED (300 mW power output, 47.3 μW/mm² maximum irradiance) for each treatment that required light irradiation.

### Assessment of In Vivo Toxicity Via H&E Staining:

BALB/c mice were treated with drug-encapsulated nanogels or saline via intravenous injection. After a one-hour circulation period, mice were euthanized, and tissue samples were harvested. The tissue samples were subsequently analyzed via H&E staining for acute toxicity.

### Example 4. Pharmaceutical Dosage Forms

The following formulations illustrate representative pharmaceutical dosage forms that may be used for the therapeutic or prophylactic administration of a composition of a formula described herein, a composition specifically disclosed herein, or a pharmaceutically acceptable salt or solvate thereof (hereinafter referred to as ‘Composition X’):

| | mg/tablet |
|---|---|
| (i) Tablet 1 | |
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/mL |
|---|---|
| (iv) Injection 1 (1 mg/mL) | |
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| | wt. % |
|---|---|
| (vii) Topical Gel 1 | |
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | |
| ‘Composition X’ | 5% |
| Methylcellulose | 2% |
| Methyl paraben | 0.2% |
| Propyl paraben | 0.02% |
| Purified water | q.s. to 100 g |
| (ix) Topical Ointment | |
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | |
| ‘Composition X’ | 5% |
| White bees wax | 10% |
| Liquid paraffin | 30% |
| Benzyl alcohol | 5% |
| Purified water | q.s. to 100 g |
| (xi) Topical Cream 2 | |
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. An NIR-light activatable nanogel comprising a crosslinked nanoparticle encapsulating a chemotherapeutic that is not covalently bound to the crosslinked nanoparticle, wherein the crosslinked nanoparticle comprises a cyanine of Formula I. 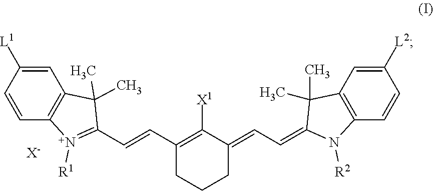
wherein
L¹ and L² each independently comprise a crosslinking moiety;
R¹ and R² are each independently —(C₁-C₆)alkyl;
X¹ is halo; and
X⁻ is a counter anion; and
a monomer crosslinked to L¹ and/or L².

2. The activatable nanogel of claim 1 wherein L¹ and L² are each independently —NR³(C═O)CR⁴═CHR⁵, —O(C═O)CR⁴═CHR⁵, or —S(C═O)CR⁴═CHR⁵; wherein R³, R⁴, and R⁵ are each independently H or —(C₁-C₆)alkyl; and each —(C₁-C₆)alkyl is independently unbranched or branched.

3. The activatable nanogel of claim 1 wherein L¹ and L² are —NH(C═O)CH═CH₂.

4. The activatable nanogel of claim 1 wherein R¹ and R² are ethyl.

5. The activatable nanogel of claim 1 wherein X¹ is chloro.

6. The activatable nanogel of claim 1 wherein X⁻ is halo.

7. The activatable nanogel of claim 1 wherein the cyanine is:

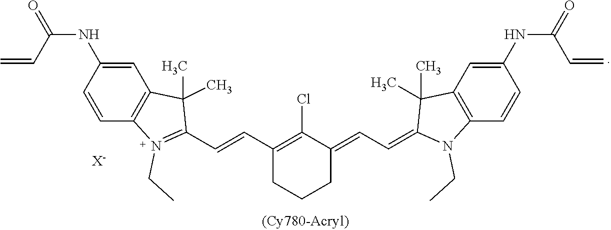

8. The activatable nanogel of claim 1 wherein the monomer is N(R³)₂(C═O)CR⁴═CHR⁵, —O(C═O)CR⁴═CHR⁵, or —S(C═O)CR⁴═CHR⁵; wherein R³, R⁴, and R⁵ are each independently H or —(C₁-C₆)alkyl; and each —(C₁-C₆)alkyl is independently branched or unbranched.

9. The activatable nanogel of claim 1 wherein the monomer is NH(CH(CH₃)₂)(C═O)CH═CH₂.

10. The activatable nanogel of claim 1 wherein the crosslinked nanoparticle has a spherical morphology and an average diameter of about 80 nanometers to about 220 nanometers.

11. The activatable nanogel of claim 1 wherein the chemotherapeutic has a molecular weight of about 300 g mol⁻¹ to about 1500 g mol⁻¹.

12. The activatable nanogel of claim 1 wherein the chemotherapeutic is paclitaxel, geftitinib, GW9662, or cisplatin.

13. A method for preparing an activatable nanogel according to claim 1, which method comprises:
a) forming an aqueous mixture comprising a surfactant, a compound of Formula I, and a monomer capable of crosslinking to L¹ and/or L² of Formula I;
b) degassing the aqueous mixture;
c) contacting the degassed aqueous mixture, a radical initiator, and a base to form a nanogel solution comprising a crosslinked nanoparticle;
d) purifying the nanogel solution; and
e) contacting the purified nanogel solution and a chemotherapeutic;
wherein the activatable nanogel is thereby formed.

14. A method for treating cancer comprising:
a) administering to a subject having a cancer an activatable nanogel according to claim 1, wherein the cancer uptakes a crosslinked nanoparticle of the activatable nanogel;
b) irradiating the cancer with near infrared light for a period of time sufficient to release a chemotherapeutic from the crosslinked nanoparticle; wherein the cancer is thereby treated.

15. The method of claim 14 wherein the irradiating forms a 1,2-dioxetane at a carbon-carbon double bond of a cyanine of Formula I, causing release of the chemotherapeutic from the crosslinked nanoparticle into the cancer.
